(19) United States
(12) Patent Application Publication
(10) Pub. No.: US 2022/0249616 A1
(43) Pub. Date: Aug. 11, 2022

(54) Osteocrin, Lebetin or ANP for Destroying Bacterial Biofilms

(71) Applicant: Université de Rouen-Normandie, Most Saint-Aignan (FR)

(72) Inventors: Olivier Lesouhaitier, Pont De L'Arche (FR); Marc Feuilloley, Le Boulay Morin (FR); Thomas Clamens, Londres (GB); Sophie Rodrigues, Evreux (FR); Sylvie Laurency, Evreux (FR); Mélissande Louis, Fourges Vexin-Sur-Epte (FR)

(21) Appl. No.: 17/629,956

(22) PCT Filed: Jul. 24, 2020
(86) PCT No.: PCT/EP2020/071029

§ 371 Date: Jan. 25, 2022

(30) Foreign Application Priority Data
Jul. 26, 2019 (FR) FR1908511

(51) Int. Cl. A61K 38/22; A61K 31/7036; A61K 38/12; A61K 31/407; A61K 31/496; A01N 63/50; A01P 1/00; A61P 31/04

(52) U.S. Cl. CPC A61K 38/2242; A61K 31/7036; A61K 38/12; A61P 31/04; A61K 31/496; A01N 63/50; A01P 1/00; A61K 31/407

(57) ABSTRACT

The present invention relates to a natriuretic peptide selected from the group made up of (i) osteocrin, an osteocrin propeptide of osteocrin derivative, (ii) a lebetin, a lebetin fragment, or a lebetin or lebetin fragment derivative, and (iii) an ANP peptide, ANP propeptide or ANP peptide derivative, for the use thereof in a method for therapeutically treating a bacterial infection associated with a bacterial biofilm in a subject, wherein said natriuretic peptide disperses the bacterial biofilm. In a particular embodiment, the natriuretic peptide is used in combination with an antibiotic.

The present invention relates to the destroying of bacterial biofilms, in particular in the context of chronic bacterial infections.

The resistance of bacteria to antibiotic therapies is widely attributed to their ability to switch from a form of planktonic life to a form of community life, called biofilm, in the tissues of the infected host. The biofilm enables bacteria to protect themselves against antimicrobial molecules and the immunity defences of the host, thereby allowing pathogens to survive in hostile environments by imparting resistance thereto against various antibacterial molecules. In addition, a small percentage of bacterial calls termed «persister cells» are held within a biofilm exposed to antibiotics and therefore become temporarily resistant and responsible for the resuming of recurrent infections.

The opportunistic pathogen Pseudomonas aeruginosa is a major cause of mortality in immunocompromised patients, in particular in patients suffering from cystic fibrosis.

The treatment of severe infections with P. aeruginosa involves several antibiotics which, for the most efficient thereof (tobramycin, colistin, ciprofloxacin, gentamicin, netilmicin and amikacin), have a narrow therapeutic index due to the existence of nephrotoxicity and ototoxicity. Additionally, over the long term these treatments generate the onset of strains resistant to antibiotics.

This is the reason why P. aeruginosa has been classified in the ESKAPE group of bacteria (Santajit and Indrawattana (2016) Biomed. Res. Int. 2016:2475067), grouping together the most critical pathogens in terms of antibiotic resistance and requiring the discovery of new molecules or therapeutic strategies. More recently, the WHO classified P. aeruginosa among the top 3 priorities regarding bacteria having critical resistance to antibiotics (to carbapenems in particular).

The present invention sets out to meet this need.

The present invention is the outcome of the unexpected discovery by the inventors that the hormone «Atrial Natriuretic Peptide» (or ANP), at a low dose (on and after 0.1 nM) and very rapidly (as soon as 30 min after administration), is capable of destroying about 80% of pre-established P. aeruginosa biofilms, and in dose-dependent manner. The inventors have also shown that other peptides belonging to the family of natriuretic peptides, more particularly osteocrin, lebetin 2α and lebetin 1β, are equally able in small doses to destroy pre-established biofilms of P. aeruginosa.

Natriuretic peptides form a family of hormones initially identified for their cardiovascular, osmoregulatory activities, for regulating blood pressure and renal sodium excretion (natriuresis, hence their name). The three chief members of this family are ANP, the «Brain Natriuretic Peptide» or BNP and «C-type Natriuretic Peptide» or CNP. The length of these peptides is between 22 and 38 amino acids and the structure of most of the members is characterized by a disulfide bridge forming a loop of 17 amino acids which imparts a so-called «omega» shape to this molecule.

It has previously been shown that ANP, BNP and CNP prevent the formation of P. aeruginosa biofilm (Desriac et al. The natriuretic peptide hormones prevent Pseudomonas aeruginosa biofilm formation through a specific bacterial target. 16th International conference on Pseudomonas, September 2017, Liverpool, United Kingdom; Rosay et al. (2015) MBio 6: 6; Desriac et al. (2018) Pathogens 7: 47).

However, no action of these peptides on already established biofilms has been demonstrated to date.

The inventors have additionally shown herein that the ability of ANP to disperse already established biofilms is not associated with or due to the capability of ANP to kill bacteria and has no effect on the intrinsic virulence of bacteria. This particular mode of action allows the preventing of emerging bacterial strains resistant to this molecule.

Also, the inventors have shown that when ANP, in small doses, is used in combination with an antibiotic such as tobramycin or ciprofloxacin also used in small doses, more than 97% of the established biofilm is destroyed.

Therefore, ANP and antibiotics together have synergic action on the destruction of bacterial biofilms.

The present invention therefore relates to a natriuretic peptide selected from the group made up of (i) osteocrin, an osteocrin propeptide or osteocrin derivative, (ii) a lebetin, a lebetin fragment, or a lebetin or lebetin fragment derivative, and (iii) an ANP peptide, ANP propeptide or ANP peptide derivative, for the use thereof in a method for therapeutically treating a bacterial infection, in particular a chronic bacterial infection, associated with a bacterial biofilm in a subject, wherein said natriuretic peptide in particular said ANP peptide, ANP propeptide or ANP peptide derivative disperses said bacterial biofilm.

In one particular embodiment, said natriuretic peptide, in particular said ANP peptide, ANP propeptide or ANP peptide derivative, is used in combination with an antibiotic.

A further object of the invention concerns a pharmaceutical composition comprising (A) a natriuretic peptide selected from the group made up of (i) osteocrin, an osteocrin propeptide or osteocrin derivative, (ii) a lebetin, a lebetin fragment, or a lebetin or lebetin fragment derivative, and (iii) an ANP peptide, ANP propeptide, or ANP peptide derivative, and (B) an antibiotic.

A further object of the invention concerns a pharmaceutical composition comprising (A) a natriuretic peptide selected from the group made up of (i) osteocrin, an osteocrin propeptide or osteocrin derivative, (ii) a lebetin, a lebetin fragment, or a lebetin or lebetin fragment derivative, and (iii) an ANP peptide, ANP propeptide or ANP peptide derivative, and (B) an antibiotic, for the use thereof in a method for therapeutically treating a bacterial infection, in particular a chronic bacterial infection, associated with a bacterial biofilm in a subject.

A further object of the invention concerns a pharmaceutical antibacterial combination comprising (A) a natriuretic peptide selected from the group made up of (i) osteocrin, an osteocrin propeptide or osteocrin derivative, (ii) a lebetin, a lebetin fragment, or a lebetin or lebetin fragment derivative, and (iii) an ANP peptide, ANP propeptide or ANP peptide derivative, and (B) an antibiotic for simultaneous, separate or sequential use in the therapeutic treatment of a bacterial infection, in particular a chronic bacterial infection, associated with a bacterial biofilm in a subject.

The present invention also concerns the in vitro or ex vivo use of a natriuretic peptide selected from the group made up of (i) osteocrin, an osteocrin propeptide or osteocrin derivative, (ii) a lebetin, a lebetin fragment, or a lebetin or lebetin fragment derivative, and (iii) an ANP peptide, ANP propeptide or ANP peptide derivative, to disperse a bacterial biofilm.

### DETAILED DESCRIPTION OF THE INVENTION

### Natriuretic Peptides

By “natriuretic peptide”, it is meant herein a member of the family of eukaryote hormones and neurohormones composed of 3 main members: the «Atrial Natriuretic Peptide» (or ANP), the «Brain Natriuretic Peptide» (or BNP) and the «C-type Natriuretic Peptide» (or CNP). These peptides are chiefly synthesized and released by the cardiomyocytes and endothelial cells. Aside from these 3 main members, the family of natriuretic peptides also includes osteocrin and «natriuretic-like» peptides such as lebetin. These various natriuretic peptides have some similarity in terms of sequence. All, except osteocrin and lebetin 1β, have a loop of 17 amino acids formed by a disulfide bridge. Among the 17 amino acids forming this loop, 11 are common to ANP and lebetin 2a. Osteocrin also has a conserved sequence signature in this region with 7 amino acids common with ANP. This conserved region of the sequence of natriuretic peptides allows the binding thereof to human natriuretic peptide receptors (NPR-A, NPR-B and NPR-C). In addition, it has been shown that ANP, osteocrin and lebetin 2α bind with strong affinity onto the AmiC sensor of P. aeruginosa (Rosay et al. (2015) mBio 25: e01033-15). On the other hand, numerous differences are observed in the amino acid sequence of their C and N terminal ends, accounting for their different physiological activities. It is of interest to note that in addition to the end active products given below, the synthesis, maturation and partial degradation of natriuretic peptides can lead to the production of numerous variants having physiological activity.

There exist many members of this family in man and in the animal kingdom: urodilatin, described below, uroguanylin, guanylin, VNP, DNP and KNP.

The ventricular natriuretic peptide (VNP) was initially identified in eels as being a peptide having similar physiological functions to those of ANP, and was later identified in numerous fish species (Kawakoshi et al. (2004) J. Mol. Endocrinol. 32:547-555).

The dendroapsis natriuretic peptide (DNP) was identified in the venom of the green mamba snake (Schweitz et al. (1992) J. Biol. Chem. 267:13928-13932) and the Krait natriuretic peptide (KNP) was identified in the venom of the Bungarus flaviceps snake (Sridharan et al. (2015) Biochem. J. 469:255-266).

Numerous other compounds similar to natriuretic peptides are regularly identified in the venom of vipers such as lebetins isolated from the venom of Macrovipera lebetina, from large reptiles such as the Komodo dragons (Natriuretic peptide toxin Var2) (Fry et al. (2009) Proc. Natl. Acad. Sci. USA 106 8 969-8974) and also from the venom of scorpions (Alves et al. (2013) Toxicon 74:19-26). They are generally called peptides with natriuretic-like activity. Among these, the preferred natriuretic peptides are L2α lebetins (Gly1-Gly38) and L1β (Asp2-Gly13). Finally, the presence is described in the bacterium Escherichia coli of an enterotoxin called ST («heat-stable enterotoxin») which has a peptide structure close to human guanylin and uroguanylin, and having natriuretic-like activity.

The natriuretic peptide used in the invention is selected from the group made up of (i) osteocrin, an osteocrin propeptide or osteocrin derivative, (ii) a lebetin, a lebetin fragment, or a lebetin or lebetin fragment derivative, and (iii) an ANP peptide, ANP propeptide or ANP peptide derivative.

By “ANP”, it is meant herein the peptide corresponding to the main mature form derived from cleavage of the N-terminal fragment of the pro-hormone proANP.

Typically, the human ANP is a peptide of 28 amino acids derived from cleavage of the N-terminal fragment of the pro-hormone proANP having 126 amino acids. Human ANP is formed of the amino acids 99 to 126 of the pro-hormone proANP.

ANP is a highly conserved peptide in its amino acid sequence since this sequence is identical between man, monkeys, gorillas, pigs, horses and sheep for example (Takei et al. (2011) General and Comparative Endocrinology 171:258-266). However, in numerous animal species in which it is expressed some differences in sequence are observed. For example, rats, mice and rabbits have a different amino acid at position 12 (Isoleucine instead of Methionine in man). Similarly, elephants have an ANP shortened to 27 amino acids and two different amino acids compared with man (Takei et al. (2011) General and Comparative Endocrinology 171:258-266).

By «ANP propeptide» it is meant herein any peptide derived from the expression of the gene encoding preproANP, from maturation and optionally successive cleavages of preproANP, and comprising the peptide sequence of ANP.

The term «ANP propeptide» therefore includes the pro-hormone proANP (typically of 126 amino acids in man) carrying ANP in its C-terminal portion, the preprohormone preproANP (typically of 151 amino acids in man) which corresponds to the protein produced in man by the NPPA gene. The preprohormone preproANP carries a peptide signal which is cleaved to form proANP.

In the context of the present invention, the term «ANP propeptide» also includes any peptide derived from maturation and from cleavages of preproANP and proANP.

ANP typically consists of the amino acid sequence SLRRSSCFGGRMDRIGAQSGLGCNSFRY (SEQ ID NO: 1), with a disulfide bridge located between the amino acids 7 and 23.

ProANP typically consists of the amino acid sequence NPMYNAVSNADLMDFKNLLDHLEEKMPLEDEVVPPQVLSEPNEEAGAALSPLPEVPPWT GEVSPAQRDGGALGRGPWDSSDRSALLKSKLRALLTAPRSLRRSSCFGGRMDRIGAQS GLGCNSFRY (SEQ ID NO: 3).

PreproANP typically consists of the amino acid sequence MSSFSTTTVSFLLLLAFQLLGQTRANPMYNAVSNADLMDFKNLLDHLEEKMPLEDEVVPP QVLSEPNEEAGAALSPLPEVPPWTGEVSPAQRDGGALGRGPWDSSDRSALLKSKLRAL LTAPRSLRRSSCFGGRMDRIGAQSGLGCNSFRY (SEQ ID NO: 4).

By «ANP derivative it is meant herein the natural or synthetic derivatives of ANP. By «ANP derivative», it is meant herein an ANP peptide such as defined above in which one or more amino acid residues have been chemically modified e.g. via alkylation, acylation, amidation, methoxylation, ester formation, amide formation, insertion of a lipophilic substituent, addition of polyethylene glycol (PEG), addition of a carbohydrate chain.

By «ANP derivative», it is also meant herein truncated forms of ANP corresponding to the amino acids 4 or 5 to 28 of the whole length of ANP, forms having a structure which is an antiparallel dimer of ANP, in particular the homodimer β-ANP, a polymer composed of ANP monomers.

By «ANP derivative», it is also meant peptide variants of ANP i.e. intermediate forms obtained during synthesis, maturation and partial degradation of ANP such as defined above, as well as ANP peptides in which 1 to 12, in particular 1 to 10, in particular 1 to 5, in particular 1 to 4, in particular 1 to 3, more particularly 1 or 2 amino acids have been deleted, substituted or added whilst preserving the activity of wild-type ANP.

In one particular embodiment, the ANP derivative is an ANP peptide in which the Phenylalanine (Phe) amino acid has been substituted in the loop at position 8 (and replaced by an Alanine amino acid for example).

In another particular embodiment, the ANP derivative is an ANP peptide in which the amino acid Phenylalanine (Phe) has been substituted at position 26 or the amino acid Serine substituted at position 25, two amino acids responsible for degradation of wild-type ANP (Ichiki and Burnett (2017) Circ J. 81: 913-919).

In one particular embodiment, the ANP derivative is a lengthened form of ANP, urodilatin having the sequence TAPRSLRRSSCFGGRMDRIGAQSGLGCNSFRY (SEQ ID NO: 2), with a disulfide bridge between the amino acids 11 and 27. This peptide corresponds to an ANP lengthened by 4 amino acids on the N-terminal side.

In another particular embodiment, the ANP derivative is a lengthened form of the ANP peptide by 12 amino acids, such as fsANP which is a form of ANP lengthened by 12 amino acids on the C-terminal side and is found in persons carrying a mutation in the gene coding for ANP (Dickey et al. (2009) J Biol Chem 284:19196-19202).

In another particular embodiment, the ANP derivative is a linearized version of ANP with cleavage of the disulfide bridge (initially located between the amino acids 7 and 23).

Typically, the ANP derivatives able to be used in the present invention are preferably modified compared with ANP so as to have a longer half-life than ANP and/or to have weaker affinity than ANP for the human endogenic receptors of ANP.

By «osteocrin», it is meant herein a hormone acting as regulator of dendritic growth in the developing cerebral cortex in response to sensory experience.

Typically, human osteocrin is a peptide of 50 amino acids derived from cleavage of the N-terminal fragment of the pro-hormone pro-osteocrin having 106 amino acids. Human osteocrin is formed of the amino acids 83 to 132 of the pro-hormone pro-osteocrin.

By «osteocrin propeptide», it is meant herein the pro-hormone pro-osteocrin having 106 amino acids and carrying osteocrin in its C-terminal portion and/or the preprohormone prepro-osteocrin having 132 amino acids which corresponds to the protein produced in man by the OSTN gene. Prepro-osteocrin carries a peptide signal which is cleaved to form pro-osteocrin having 106 amino acids.

Osteocrin typically consists of the amino acid sequence SFSGFGSPLDRLSAGSVDHKGKQRKWDHPKRRFGIPMDRIGRNRLSNSR (SEQ ID NO: 5).

Pro-osteocrin typically consists of the amino acid sequence VDVTTTEAFDSGVIDVQSTPTVREEKSATDLTAKLLLLDELVSLENDVIETKKKRSFSGFG SPLDRLSAGSVDHKGKQRKWDHPKRRFGIPMDRIGRNRLSNSRG (SEQ ID NO: 6).

Prepro-osteocrin typically consists of the amino acid sequence MLDWRLASAHFILAVTLTLWSSGKVLSVDVTTTEAFDSGVIDVQSTPTVREEKSATDLTA KLLLLDELVSLENDVIETKKKRSFSGFGSPLDRLSAGSVDHKGKQRKVVDHPKRRFGIPM DRIGRNRLSNSRG (SEQ ID NO: 7).

In the context of the present invention, the term «osteocrin propeptide» also includes any peptide derived from maturation and cleavages of prepro-osteocrin and pro-osteocrin.

By «osteocrin derivative», it is meant herein natural or synthetic derivatives of osteocrin.

By «osteocrin derivative», it is meant herein an osteocrin such as defined above in which one or more amino acid residues have been chemically modified e.g. via alkylation, acylation, amidation, methoxylation, ester formation, amide formation, insertion of a lipophilic substituent, addition of polyethylene glycol (PEG), addition of a carbohydrate chain.

By «osteocrin derivative», it is also meant peptide variants of osteocrin i.e. intermediate forms obtained during synthesis, maturation and partial degradation of osteocrin such as defined above.

By «lebetin», it is meant herein a peptide derived from the venom of the Macrovipera lebetin viper.

The lebetin such as defined above is preferably lebetin 2α or lebetin 1β.

By «lebetin 2α», it is meant herein a peptide produced by the Macrovipera lebetina viper described in Barbouche et al. (1996) FEBS Lett. 392:6-10.

Typically, lebetin 2α is a peptide of 38 amino acids.

Lebetin 2α typically consists of the amino acid sequence GDNKPPKKGPPNGCFGHKIDRIGSHSGLGCNKVDDNKG (SEQ ID NO: 8) with a disulfide bridge located between amino acids 14 and 30.

By «lebetin 1β», it is meant herein a peptide produced by the Macrovipera lebetina viper described in Barbouche et al. (1998) Toxicon 36(12): 1939-47.

Typically, lebetin 1β is a peptide of 12 amino acids.

Lebetin 1β typically consists of the amino acid sequence DNKPPKKGPPNG (SEQ ID NO: 9).

By «lebetin fragment», it is designated herein a fragment of at least 10 amino acids of said lebetin, preferably of lebetin 2α such as defined above or of lebetin 1β such as defined above.

Said lebetin fragment preferably comprises at least 10 amino acids, preferably at least 12 amino acids, preferably at least 14 amino acids, preferably at least 16 amino acids and/or no more than 30 amino acids, preferably no more than 25 amino acids, preferably no more than 20 amino acids.

A fragment of lebetin 2α comprises for example the amino acids 4 to 10 of sequence SEQ ID NO: 8, preferably the amino acids 2 to 12 of sequence SEQ ID NO: 8.

A fragment of lebetin 2α consists for example of the amino acids 1 to 13 of sequence SEQ ID NO: 8.

Lebetin 1β of sequence SEQ ID NO: 9 is also a fragment of lebetin 2α consisting of the amino acids 2 to 13 of sequence SEQ ID NO: 8.

By «lebetin or lebetin fragment derivative», it is meant herein natural or synthetic derivatives of lebetin or of a lebetin fragment.

By «lebetin or lebetin fragment derivative», it is meant herein a lebetin such as defined above or a lebetin fragment such as defined in which one or more amino acid residues have been chemically modified e.g. via alkylation, acylation, amidation, methoxylation, ester formation, amide formation, insertion of a lipophilic substituent, addition of polyethylene glycol (PEG), addition of a carbohydrate chain.

By «lebetin or lebetin fragment derivative», it is also meant peptide variants of lebetin or of a lebetin fragment i.e. intermediate forms obtained during synthesis, maturation and partial degradation of lebetin or of a lebetin fragment such as defined above.

By «other natriuretic peptide», it is meant herein a natriuretic peptide which is not ANP, an ANP propeptide or ANP derivative (such as urodilatin).

### Bacterial Biofilm and Bacterial Infection

By «bacterial infection», it is meant herein an infection due to one or more species of bacteria, in particular Gram-negative and/or Gram-positive bacteria.

In one particular embodiment the bacterial infection is a chronic bacterial infection.

In one particular embodiment, the bacterial infection is an infection with a Gram-negative bacterium.

In one particular embodiment, the bacterial infection is an infection with a bacterium of genus Pseudomonas, more particularly the species Pseudomonas aeruginosa.

In another particular embodiment, the bacterial infection is an infection with a Gram-positive bacterium.

In one particular embodiment, the bacterial infection is an infection with a bacterium of genus Staphylococcus, more particularly the species Staphylococcus aureus.

By «bacterial biofilm», it is meant herein a community of bacteria adhering together and attached to a surface. Biofilm production is characterized by the secretion of a matrix which is able to attach to numerous types of surfaces, allowing cohesion and protection of the bacteria forming this structure.

In the invention, a bacterial biofilm comprises at least the one or more bacterial species responsible for the infection to be treated.

In the invention, said natriuretic peptide, in particular said ANP peptide, ANP propeptide or ANP peptide derivative, disperses the bacterial biofilm.

By «dispersion of the bacterial biofilm», or «destruction of the bacterial biofilm», it is meant herein that the bacteria forming the constituent community of the bacterial biofilm separate from each other and from the surface, so that the biofilm is reduced and even disappears, typically providing antibiotics with new access to these bacteria.

In one particular embodiment, said bacterial biofilm is dispersed by at least 50%, in particular at least 55%, in particular at least 60%, in particular at least 65%, in particular at least 70%, at least 75%, at least 80%, at least 85%, at least 90%, at least 95% or at least 100%.

Advantageously, said natriuretic peptide, in particular said ANP peptide, ANP propeptide or ANP peptide derivative, disperses the bacterial biofilm without killing the constituent bacteria of this biofilm.

It is to be noted however, that when said natriuretic peptide, in particular said ANP peptide, ANP propeptide or ANP peptide derivative, is used in combination with an antibiotic, the constituent bacteria of the biofilm can be killed by said antibiotic, in particular when they are released further to the dispersive effect of the natriuretic peptide, in particular ANP, an ANP propeptide or ANP peptide derivative.

### Therapeutic Application

The present invention concerns a natriuretic peptide selected from the group made up of (i) osteocrin, an osteocrin propeptide or osteocrin derivative, (ii) a lebetin (preferably lebetin 2α or lebetin 1β), a lebetin fragment, or a lebetin or lebetin fragment derivative, and (iii) an ANP peptide, ANP propeptide or ANP peptide derivative such as defined under the foregoing section «Natriuretic peptides» for use thereof in a method for therapeutically treating a bacterial infection associated with a bacterial biofilm such as defined under the foregoing section «Bacterial biofilm and bacterial infection» in a subject, wherein said natriuretic peptide disperses said bacterial biofilm.

The present invention also concerns the use of a natriuretic peptide selected from the group made up of (i) osteocrin, an osteocrin propeptide or osteocrin derivative, (ii) a lebetin (preferably lebetin 2α or lebetin 1β), a lebetin fragment, or a lebetin or lebetin fragment derivative, and (iii) an ANP peptide, ANP propeptide or ANP peptide derivative such as defined under the foregoing section «Natriuretic peptides» for the manufacture of a medication for the therapeutic treatment of a bacterial infection associated with a bacterial biofilm such as defined under the foregoing section «Bacterial biofilm and bacterial infection» in a subject, wherein said natriuretic peptide disperses said bacterial biofilm.

The present invention also concerns a method for therapeutically treating a bacterial infection associated with a bacterial biofilm such as defined under the foregoing section «Bacterial biofilm and bacterial infection» in a subject suffering from a bacterial infection, in particular a chronic bacterial infection, comprising the administering to said subject of a therapeutically effective amount of a natriuretic peptide selected from the group made up of (i) osteocrin, an osteocrin propeptide or osteocrin derivative, (ii) a lebetin (preferably lebetin 2α or lebetin 1β), a lebetin fragment, or a lebetin or lebetin fragment derivative, and (iii) an ANP peptide ANP propeptide or ANP peptide derivative such as defined under the foregoing section «Natriuretic peptides», wherein said natriuretic peptide disperses said bacterial biofilm.

By «subject», it is meant herein a human or an animal such as a non-human mammal, a bird or fish.

In one particular embodiment, the subject suffers from a chronic respiratory pathology such as cystic fibrosis, chronic obstructive pulmonary disease, or bronchiectasis.

Preferably the subject suffers from cystic fibrosis.

In another particular embodiment, said subject suffers from major burns (serious burn victim) or from a surface infection.

In another embodiment, said subject carries an implant and/or a prosthesis.

In one particular embodiment, said natriuretic peptide, in particular said ANP peptide, ANP propeptide or ANP peptide derivative, is used in combination with an antibiotic.

By «antibiotic», it is meant herein a natural or synthetic substance destroying or blocking bacterial growth.

Antibiotics include:
Antibiotics inhibiting bacterial cell wall synthesis, among which:
   beta-lactams, in particular:
      penicillins including group A penicillins such as amoxicillin; group G and V penicillins such as benzathine benzylpenicillin, benzathine penicillin, benzathine phenoxymethylpenicillin, penicillins G and penicillin V; group M penicillins such as cloxacillin and oxacillin; carboxypenicillins such as ticarcillin; ureidopenicillins such as piperacillin; aminidopenicillins such as pivmecillinam; and temocillin;
      carbapenems such as ertapenem, imipenem and meropenem;
      monobactams such as aztreonam;
      cephalosporins including 1^{st} generation cephalosporins such as cefaclor, cefadroxil, cephalexin, cephalothin, cefazolin and cephradine; 2^{nd} generation cephalosporins such as cefamandole, cefoxitin, cefuroxime sodium and cefuroxime axetil; and 3^{rd} generation cephalosporins such as cefixime, cefpodoxime proxetil, cefotiam hexetil, cefepime, cefotaxime, cefpirome, ceftazidime and ceftriaxone;
   fosfomycins such as fosfomycin and fosfomycin trometamol;
   glycopeptides such as teicoplanin and vancomycin;
   lipopeptides such as daptomycin; and
   polymyxins such as polymyxin B and polymyxin E or colistin;
antibiotics inhibiting protein synthesis, among which:
   aminoglycosides such as amikacin sulfate, gentamicin, neomycin, netilmicin, spectinomycin, streptomycin and tobramycin;
   macrolides and the like, in particular:
      true macrolides such as amphotericin B, azithromycin, clarithromycin, erythromycin, josamycin, midecamycin, roxithromycin;
      lincosamides such as clindamycin and lincomycin;
      ketolides such as telithromycin; and
      synergistins such as pristinamycin;
   phenicols such as thiamphenicol;
   cyclines such as chlortetracycline, doxycycline, lymecycline, methacycline, minocycline and tigecycline;
   fusidic acids such as fusidic acid; and
   oxazolidinones such as linezolid and tedizolid;
antibiotics inhibiting the synthesis of nucleic acids, among which:
   quinolones such as pipemidic acid, flumequine, enoxacin, lomefloxacin, norfloxacin, ciprofloxacin, ofloxacin, pefloxacin, levofloxacin and moxifloxacin;
   quinolines such as hydroxyquinoline;
   mupirocin; and
   rifamycin;
antibiotics inhibiting the synthesis of folic acid including sulfamides such as sulfadiazine, sulfamethizole, sulfafurazole and sulfamethoxazole; and
antibiotics having complex or ill-known mechanisms, among which:
   nitro products, in particular:
      nitrofurans such as nitrofurantoin and nifuroxazide; and
      nitro-imidazoles such as metronidazole, omidazole and tinidazole; and
   ethambutol, isoniazid, pyrazinamide, rifabutin and rifampicin.

In one particular embodiment, said antibiotic is an antibiotic inhibiting protein synthesis, in particular an aminoglycoside such as tobramycin; an antibiotic inhibiting the synthesis of nucleic acid, in particular a quinolone such as ciprofloxacin; an antibiotic inhibiting bacterial cell wall synthesis, in particular a beta-lactam, more particularly a carbapenem such as imipenem, doripenem or meropenem; or an antibiotic acting on the structure of the bacterial cell wall in particular a polymyxin such as polymyxin B or colistin.

In one particular embodiment, said antibiotic is an aminoglycoside antibiotic, a quinolone, a carbapenem or polymyxin. More particularly, said antibiotic can be an aminoglycoside or a quinolone.

In another particular embodiment, said antibiotic is tobramycin, ciprofloxacin, imipenem and/or colistin.

More particularly, said antibiotic can be tobramycin and/or ciprofloxacin.

The inventors have shown that the combined use of the ANP peptide with these antibiotics has a synergic effect on the destruction of biofilms. Without being bound by theory, it is assumed that the ANP peptide, by dispersing the constituent bacteria of the biofilm, makes these bacteria more accessible to antibiotics which are therefore more effective.

In another particular embodiment, said natriuretic peptide is used in combination with at least one other natriuretic peptide such as defined under the section «Natriuretic peptides».

Therefore, in one particular embodiment, when said natriuretic peptide is ANP, an ANP propeptide or ANP peptide derivative, it can be used in combination with osteocrin, an osteocrin propeptide or osteocrin derivative and/or with a lebetin (preferably lebetin 2α or lebetin 1β), a lebetin fragment, or a lebetin or lebetin fragment derivative.

Alternatively in another particular embodiment when said natriuretic peptide is osteocrin, an osteocrin propeptide or osteocrin derivative, it can be used in combination with ANP, an ANP propeptide or ANP peptide derivative, and/or with a lebetin (preferably lebetin 2α or lebetin 1β), a lebetin fragment, or a lebetin or lebetin fragment derivative.

Alternatively, in another particular embodiment, when said natriuretic peptide is lebetin (preferably lebetin 2α or lebetin 1β), a lebetin fragment, or a lebetin or lebetin fragment derivative, it can be used in combination with ANP, an ANP propeptide or ANP peptide derivative and/or with osteocrin, an osteocrin propeptide or osteocrin derivative.

The combined use of several natriuretic peptides has the advantage of being able to use smaller doses of each of the compounds, thereby further efficaciously limiting any possible side effects

In this particular embodiment, said natriuretic peptide can also be used in combination with an antibiotic as described above, optionally sequentially (first said natriuretic peptide followed by the antibiotic).

In one particular embodiment, said natriuretic peptide, in particular said ANP peptide, ANP propeptide or ANP peptide derivative, is used in combination with any other substance adapted for treating the subject suffering from the bacterial infection such as described above, optionally sequentially (first said natriuretic peptide followed by the adapted substance).

In particular, when the subject suffers from a chronic respiratory pathology, said natriuretic peptide, in particular said ANP peptide, ANP propeptide or ANP peptide derivative, is preferably used in combination with a substance adapted for the treatment of this chronic respiratory pathology, optionally sequentially (first said natriuretic peptide followed by the adapted substance).

Therefore, in the embodiment in which said subject suffers from cystic fibrosis, said natriuretic peptide, in particular said ANP peptide, ANP propeptide or ANP peptide derivative, is preferably used in combination with a substance for mucociliary clearance such as a Dnase (e.g. Pulmozyme®), and/or a bronchodilator, optionally sequentially (first a substance for mucociliary clearance followed by said natriuretic peptide).

In these particular embodiments, said natriuretic peptide can also be used in combination with an antibiotic as described above and/or with another natriuretic peptide as described above, preferably with an antibiotic as described above.

In the context of the invention, the term «treatment» or «to treat» means to reverse, relieve or inhibit the progress of the disease to which this term is applied, or one or more symptoms of this disease.

By «therapeutically effective amount», it is meant herein a sufficient amount of the compound of interest to disperse the bacterial biofilm when treating the bacterial infection, with a reasonable risk-benefit ratio applicable to any medical treatment. It will be clearly understood however that the total daily use of the compounds employed in the invention will be decided by medical practitioners on the basis of their medical assessment. The level of therapeutically effective dose specific to a particular subject will depend upon a certain number of factors including the disease to be treated and the seriousness thereof, the activity of the compounds specifically employed, age, weight, general state of health, gender and diet, administration time, route of administration and rate of excretion of the specific compounds administered, length of treatment, the medications used in combination or coincidentally with the specific compounds administered, and similar factors well known in the medical sphere. For example, it is well known in this sphere to start with doses of compounds at lower levels than those required to reach the desired therapeutic effect, and gradually to increase dosage until the desired effect is obtained.

The compounds used in the context of the invention can be administered in the form of a pharmaceutical composition comprising pharmaceutically acceptable excipients, and optionally delayed release matrices such as biodegradable polymers to form therapeutic compositions.

By «pharmaceutical» or «pharmaceutically acceptable», it is meant herein molecular entities and compositions which do not produce allergic or other undesirable secondary reactions when administered to a mammal, in particular to a human. A pharmaceutically acceptable vehicle or excipient designates a solid, semi-solid or liquid nontoxic filler, diluent, encapsulating material or a formulation auxiliary of any type.

The form of the pharmaceutical compositions comprising the compounds used in the context of the invention and the route of administration will evidently depend upon the disease to be treated, the severity of the disease, patient age, weight and gender, etc. . . . .

The compounds used in the context of the invention can be formulated for administration via airway route or inhalation (in particular by nebulization), via oral, parenteral, intranasal, intravenous, intramuscular, topical, sub-cutaneous or intraocular route for example.

In one particular embodiment, the compounds used in the context of the invention are administered via airway route or inhalation (in particular by nebulization).

In one particular embodiment, the natriuretic peptide in particular the ANP peptide (or propeptide or derivative) used in the context of the invention is administered at a daily dose of 0.3 ng/ml to 3.000 ng/ml.

In another particular embodiment, the antibiotic used in combination is administered at a maximum daily dose of 600 mg (300 mg twice a day).

### Pharmaceutical Composition and Pharmaceutical Combination

The present invention also concerns a pharmaceutical composition comprising (A) a natriuretic peptide selected from the group made up of (i) osteocrin, an osteocrin propeptide or osteocrin derivative, (ii) a lebetin (preferably lebetin 2α or lebetin 1β) a lebetin fragment, or a lebetin or lebetin fragment derivative and (iii) an ANP peptide, ANP propeptide or ANP peptide derivative such as defined under the foregoing section «Natriuretic peptides» and (B) an antibiotic such as defined under the foregoing section «Therapeutic application».

In one particular embodiment, the composition of the invention further comprises a pharmaceutically acceptable vehicle or excipient such as defined above.

In another particular embodiment, the composition of the invention further comprises another natriuretic peptide such as defined under the foregoing section «Natriuretic peptides».

The present invention also concerns a pharmaceutical antibacterial combination comprising (A) a natriuretic peptide selected from the group made up of (i) osteocrin, an osteocrin propeptide or osteocrin derivative, (ii) a lebetin (preferably lebetin 2α or lebetin 1β), a lebetin fragment, or a lebetin or lebetin fragment derivative and (iii) an ANP peptide, ANP propeptide or ANP peptide derivative such as defined under the foregoing section «Natriuretic peptides» and (B) an antibiotic such as defined under the foregoing section «Therapeutic application» for simultaneous, separate or sequential use in the therapeutic treatment of a subject such as defined above, for a bacterial infection associated with a bacterial biofilm such as defined under the foregoing section «Bacterial biofilm and bacterial infection».

The present invention also concerns the use (A) of a natriuretic peptide selected from the group made up of (i) osteocrin, an osteocrin propeptide or osteocrin derivative, (ii) a lebetin (preferably lebetin 2α or lebetin 1β), a lebetin fragment, or a lebetin or lebetin fragment derivative and (iii) an ANP, peptide, ANP propeptide or ANP peptide derivative such as defined under the foregoing section «Natriuretic peptides» and (B) an antibiotic such as defined under the foregoing section «Therapeutic application» for the manufacture of a pharmaceutical antibacterial combination for simultaneous, separate or sequential administration in the therapeutic treatment of a subject such as defined above for a bacterial infection associated with a bacterial biofilm such as defined under the foregoing section «Bacterial biofilm and bacterial infection».

A further object of the present invention is a method for therapeutically treating a bacterial infection associated with a bacterial biofilm such as defined under the foregoing section «Bacterial biofilm and bacterial infection», comprising the simultaneous, separate or sequential administration in a subject in need thereof of a therapeutically effective amount of (A) a natriuretic peptide selected from the group made up of (i) osteocrin, an osteocrin propeptide or osteocrin derivative, (ii) a lebetin (preferably lebetin 2α or lebetin 1β), a lebetin fragment, or a lebetin or lebetin fragment derivative and (iii) an ANP peptide, ANP propeptide or ANP peptide derivative such as defined under the foregoing section «Natriuretic peptides» and (B) an antibiotic such as defined under the foregoing section «Therapeutic application».

In the context of the invention, the term «combination» or «pharmaceutical combination» defines either a fixed combination in a single unit dose form, or a kit for combined administration in which the natriuretic peptide and the antibiotic can be administered independently at the same time or separately at time intervals allowing the combination partners to exhibit a synergic effect.

The compounds of the combination of the invention can therefore be formulated in one or two separate pharmaceutical combinations, each composition to be given via the same route of administration or via different routes of administration.

In one particular embodiment, said pharmaceutical combination of the invention also comprises (C) another natriuretic peptide such as defined under the section «Natriuretic peptides», and/or (D) another substance adapted for treatment of the subject suffering from the bacterial infection such as defined under the foregoing section «Therapeutic application».

In this particular embodiment, the combination can be a fixed combination in a single unit dose form, or a kit for combined administration in which the natriuretic peptide, the antibiotic, other natriuretic peptide and/or other substance adapted for the treatment of the subject can be administered independently at the same time or separately at time intervals allowing the combination partners to exhibit a synergic effect.

In this embodiment, the compounds of the combination can therefore be formulated in one, two, three or four separate pharmaceutical combinations, each composition being for the same route of administration or for different routes of administration.

To prepare the pharmaceutical compounds used in the invention, an effective amount of the compounds used in the invention can be dissolved or dispersed in a pharmaceutically acceptable vehicle or aqueous medium.

The pharmaceutical forms adapted for administration via injection include sterile aqueous solutions or dispersions and sterile powders for extemporaneous preparation of sterile injectable solutions or dispersions, for example in micronized form. Whichever the case, the form must be sterile and must be fluid for easy use with a syringe. It must be stable under conditions of manufacture and storage and must be protected against the contaminating action of microorganisms such as bacteria, viruses or fungi.

The vehicle can be a solvent or dispersing medium for example comprising water, ethanol, a polyol (e.g. glycerol, propylene glycol, liquid polyethylene glycol, or other), and suitable mixtures thereof. Suitable fluidity can be maintained for example by using a coating such as lecithin, by maintaining a required particle size for dispersions, and by using surfactants, stabilizing agents, cryoprotectants or anti-oxidants. The prevention of action by microorganisms can be provided by antibacterial and antifungal agents. In many cases, it will be preferable to include isotonic agents e.g. sugars or sodium chloride.

Sterile injectable solutions can be prepared by incorporating the active compounds in a required amount of suitable solvent with several of the other ingredients, followed by filter sterilization. In general, the dispersions are prepared by incorporating the various sterilized active ingredients in a sterile vehicle containing the basic dispersion medium and the other required ingredients. With regard to sterile powders for preparing sterile injectable solutions, the preferred preparation methods are the techniques of vacuum drying and freeze-drying, which produce a powder of the active ingredient plus any desired additional ingredient from a solution thereof previously sterilized by filtration.

For parenteral administration in an aqueous solution for example, the solution is preferably suitably buffered if necessary, and the liquid diluent made isotonic with sufficient saline or glucose solution. These particular aqueous solutions are especially suitable for intravenous, intramuscular, sub-cutaneous or intraperitoneal administration.

The formulations of pharmaceutical compositions for administration via inhalation are well known to those skilled in the art. In general, the active ingredients are delivered in the form of an aerosol spray from a pressurized metered-dose inhaler which uses a suitable propellant gas such as dichlorodifluoromethane, trichlorofluoromethane, dichloro-tetrafluoroethane or carbon dioxide, in the form of a powder administered by a dry powder inhaler or in the form of an aqueous liquid aerosol using a nebulizer. Nebulizers for the delivery of a liquid aerosol can be categorized into jet nebulizers applying a pressurized flow of air using a portable compressor or central air supply in a hospital, ultrasonic nebulizers incorporating a piezo-crystal providing the energy needed to generate aerosolization, and electronic nebulizers based on the principle of a perforated vibrating membrane.

### Non-Therapeutic Uses

The present invention also concerns the in vitro or ex vivo use of a natriuretic peptide selected from the group made up of i) osteocrin, an osteocrin propeptide or osteocrin derivative, (ii) a lebetin (preferably lebetin 2α or lebetin 1β), a lebetin fragment, or a lebetin or lebetin fragment derivative and (iii) an ANP peptide, ANP propeptide or ANP peptide derivative such as defined under the foregoing section «Natriuretic peptides», to disperse a bacterial biofilm.

The natriuretic peptide, in particular ANP, ANP propeptide or ANP peptide derivative can therefore typically be used in antifouling applications e.g. on ship hulls or in pipelines.

The present invention therefore also concerns an in vitro or ex vivo method for dispersing a bacterial biofilm on a surface, comprising the application to said surface of a composition comprising a natriuretic peptide selected from the group made up of (i) osteocrin, an osteocrin propeptide or osteocrin derivative, (ii) a lebetin (preferably lebetin 2α or lebetin 1β), a lebetin fragment, or a lebetin or lebetin fragment derivative, and (iii) an ANP peptide, ANP propeptide of ANP peptide derivative, such as defined under the foregoing section «Natriuretic peptides».

Said surface can typically be a pipe surface, ship surface, the surface of an implantable device before implantation.

The application of said composition to said surface can be implemented using any suitable technique depending upon the formulation of the composition.

The present invention is described in more detail in the examples and Figures below.

### BRIEF DESCRIPTION OF THE SEQUENCES

| SEQ ID | | |
|---|---|---|
| NO | Description | Sequence |
| 1 | Amino acid sequence of ANP | SLRRSSCFGGRMDRIGAQSGLGCNSFRY |
| 2 | Amino acid sequence of urodilatin | TAPRSLRRSSCFGGRMDRIGAQSGLGCN |
| | | SFRY |
| 3 | Amino acid sequence of proANP | NPMYNAVSNADLMDFKNLLDHLEEKMPL |
| | | EDEVVPPQVLSEPNEEAGAALSPLPEVP |
| | | PWTGEVSPAQRDGGALGRGPWDSSDR |
| | | SALLKSKLRALLTAPRSLRRSSCFGGRM |
| | | DRIGAQSGLGCNSFRY |
| 4 | Amino acid sequence of preproANP | MSSFSTTTVSFLLLLAFQLLGQTRANPMY |
| | | NAVSNADLMDFKNLLDHLEEKMPLEDEV |
| | | VPPQVLSEPNEEAGAALSPLPEVPPWTG |
| | | EVSPAQRDGGALGRGPWDSSDRSALLK |
| | | SKLRALLTAPRSLRRSSCFGGRMDRIGA |
| | | QSGLGCNSFRY |
| 5 | Amino acid sequence of osteocrin | SFSGFGSPLDRLSAGSVDHKGKQRKVVD |
| | | HPKRRFGIPMDRIGRNRLSNSR |
| 6 | Amino acid sequence of pro- | VDVTTTEAFDSGVIDVQSTPTVREEKSAT |
| | osteocrin | DLTAKLLLLDELVSLENDVIETKKKRSFSG |
| | | FGSPLDRLSAGSVDHKGKQRKVVDHPKR |
| | | RFGIPMDRIGRNRLSNSRG |
| 7 | Amino acid sequence of prepro- | MLDVVRLASAHFILAVTLTLWSSGKVLSVD |
| | osteocrin | VTTTEAFDSGVIDVQSTPTVREEKSATDL |
| | | TAKLLLLDELVSLENDVIETKKKRSFSGFG |
| | | SPLDRLSAGSVDHKGKQRKVVDHPKRRF |
| | | GIPMDRIGRNRLSNSRG |
| 8 | Amino acid sequence of lebetin 2α | GDNKPPKKGPPNGCFGHKIDRIGSHSGL |
| | | GCNKVDDNKG |
| 9 | Amino acid sequence of lebetin 1β | DNKPPKKGPPNG |

### BRIEF DESCRIPTION OF THE FIGURES

FIG. 1 gives graphs representing the growth of P. aeruginosa in the presence of different concentrations of ANP.

### EXAMPLES

### Example 1: Effect of ANP Alone (0.1 μM) on Biofilms in Formation, on Preformed Biofilms of P. aeruginosa and on the Growth of P. aeruginosa

This example shows the action of ANP on pre-formed biofilms of P. aeruginosa.

### Material and Methods

### Tested Substances, Bacterial Strains and Bacterial Cultures

The P. aeruginosa wild-type strain PA14 used was supplied by the Harvard Medical School (Boston, Mass.) (Liberati et al. (2006) Proc. Natl. Acad. Sci. USA 103: 2833-2838).

The bacterial strains were cultured at 37° C. in Luria Bertani medium (LB) under agitation.

Formation of the P. aeruginosa Biofilm Under Dynamic Conditions

After 3 h pre-culture in LB medium at 37° C., P. aeruginosa was inoculated at OD₆₀₀=0.08 in LB medium and sub-cultured for 2 h.

The ANP (Calbiochem Merck) was added 2 h after the start of culture, a time corresponding to the middle of the exponential growth phase of the bacteria. Final bacterial density and absence of contamination were controlled by seeding.

The biofilms were formed under dynamic conditions at 37° C. in a 3-channel flow chamber as described in Bazire et al. (2010) J. Bacteriol. 192:3001-3010. Briefly, from an 18 h pre-culture, a bacterial suspension at OD₆₀₀=0.08 prepared in sterile physiological saline solution was injected into each channel of the flow chamber. The bacteria were left for 2 h at 37° C. under static conditions (no flow) so that they adhered to the glass slide. Study of biofilm formation was then conducted under a flow of LB medium (2.5 ml/h) for 24 h at 37° C.

Destruction of the P. aeruginosa Biofilm Under Dynamic Conditions.

To study the impact of ANP on 24 h pre-formed biofilms, 300 μl of ANP (0.1 μM) or 300 μl of sterile ultra-pure water (control conditions) were injected into different channels of the flow chamber. Treatment of the preformed biofilm was conducted for 2 h at 37° C. under static conditions. After the 2 h, the flow of medium was again applied for 15 min to remove any cells which may have detached from the biofilm under action of the treatment.

The biofilms were observed under a confocal laser scanning microscope (Zeiss LSM710 (Zeiss) after being stained for 15 min with 5 μM of Syto9 Green fluorochrome (Invitrogen). Images were taken in the different layers of the biofilm allowing three-dimensional reconstitution; at least 3 images were taken at different points of one same channel of the flow chamber. The files of each image were then analysed with COMSTAT software (Heydorn et al. (2000) Microbiology 146:2395-2407); 3 images of at least 3 independent experiments were analysed allowing determination of the average and maximum thicknesses of the biofilms and of bacterial biovolume.

### Cell Culture

The line of human pulmonary epithelial cells of type II A549 (ATCC-CCL185TM, ATCC Manassas, Va.) was placed in culture at 37° C. under 5% CO₂ atmosphere in DMEM medium (Lonza) supplemented with 10% foetal calf serum (Lonza) and 1% penicillin and streptomycin (Penistrep, Lonza). Under routine cell culture, the cells were seeded in a 25 ml flask and used at 80% confluence.

For cytotoxicity tests, the cells were seeded in 24-well plates to a final density of 3×10⁵ cells per well and cultured 48 h before use. For a minimum time of 24 h before the infection tests, the cells were deprived of antibiotics and foetal calf serum through the addition of a medium without fresh serum.

### Measurement of the Release of Cytosolic Lactate Dehydrogenase (LDH) by A549 Cells

LDH is a stable cytosolic enzyme released into the culture medium after cell lysis and hence a marker of cell death. The amount of LDH released by the eukaryote cells in the presence of bacteria, whether or not exposed to ANP (concentration of 1 μM at 10 nM), was determined using the Cytotox 96 enzymatic test (Promega, Charbonnières, France). The A549 cells were incubated for 6 h on a control (without treatment) or with P. aeruginosa PA14 (pre-treated with ANP) with a multiplicity of infection of 10. A lysis buffer consisting of Triton X-100 solution (9% in water), was employed to determine the maximum LDH potentially released by the A549 cells under the experimental conditions (100% LDH release). A level of background noise was determined using the culture medium alone and defined as 0% LDH release, to exclude contribution by the culture medium. Percentage LDH release in the cell population was then calculated with the equation:

$$\% L D H = \frac{OD sample}{OD 100 \%} \times 100$$

The test was sufficiently sensitive to measure an LDH concentration equivalent to lysis of 1 of the cell population.

### Results

Effect of ANP on a Preformed Biofilm of P. aeruginosa

The inventors examined the potential anti-biofilm activity of ANP on an established biofilm of P. aeruginosa. From this perspective, 24 h after the formation of a P. aeruginosa biofilm in a dynamic flow system, the inventors exposed the bacteria to ANP for 2 h at 0.1 μM. Under these conditions, the inventors observed dispersion of the biofilm which had been perturbed after exposure to ANP and absence of the mushroom-shaped structures seen under the control conditions and characteristic of a P. aeruginosa biofilm. The bacterial biomass was reduced after exposure to ANP for 2 hours at 0.1 μM by 81.4±4.1% (P<0.001) compared with the biomass present under the control conditions. In parallel, the inventors observed a major reduction in the thickness of the biofilm after 2 h exposure to ANP.

Similar results were obtained when the preformed biofilm was exposed to ANP for 30 minutes. Under these conditions, the inventors observed that the biofilm is strongly dispersed by ANP, and the mushroom-shaped structures observed under the control conditions and characteristic of a P. aeruginosa biofilm, were absent after exposure to ANP. The remaining bacterial biomass was reduced after exposure to ANP (30 minutes) by 80.2±2.0% (P<0.05) compared with the biomass present under the control conditions.

Effect of ANP on the Growth of P. aeruginosa

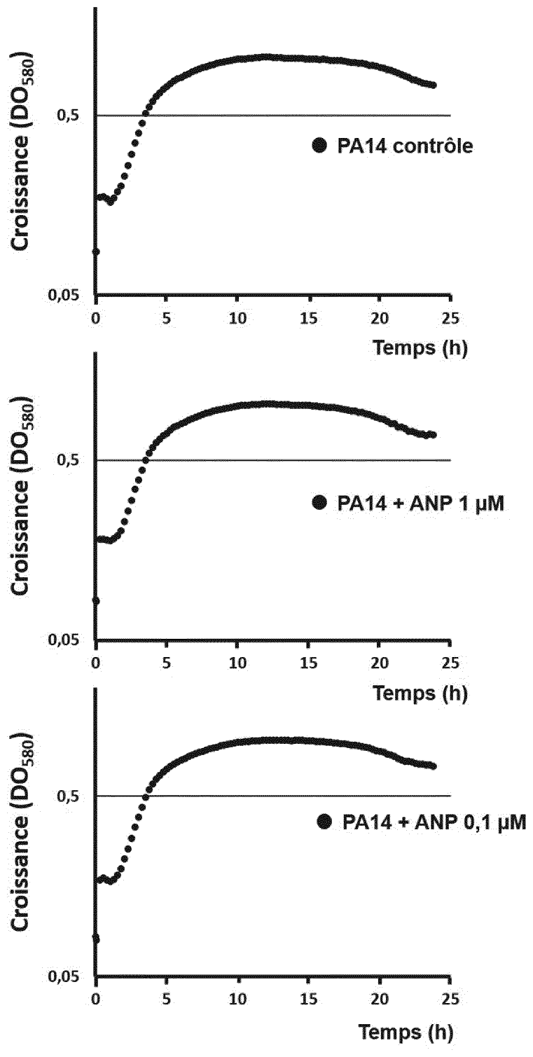

To determine a potential direct effect of ANP on the growth of P. aeruginosa, the inventors studied the impact of ANP at 1 μM and 0.1 μM on the growth of P. aeruginosa P14 in a liquid medium. None of the tested ANP concentrations affected bacterial growth as shown in FIG. 1.

Therefore, the effect of ANP on preformed biofilms cannot be attributable to action on growth of the bacterium.

### Effect of ANP on Bacterial Cytotoxicity in Cultured Human Pulmonary Cells.

The cytotoxic activity of P. aeruginosa exposed to different concentrations of ANP (1 μM to 10 nM) was studied on A549 pulmonary cells. The inventors observed that ANP, at any concentration, had no impact on the cytotoxic activity of P. aeruginosa in pulmonary cells, as shown in the Table below.

| | % LDH |
|---|---|
| Experimental conditions | (dead cells) |
| PA14 non-treated | 56.4 ± 5.5 |
| PA14 + ANP (10⁻⁶ M) | 58.9 ± 5.8 |
| PA14 + ANP (10⁻⁷ M) | 53.5 ± 5.3 |
| PA14 + ANP (10⁻⁸ M) | 54.5 ± 5.5 |

### Conclusion

The inventors have therefore shown in this example that ANP, used at a concentration of 0.1 μM, fully prevents the formation of a P. aeruginosa biofilm and strongly destroys a preformed biofilm of P. aeruginosa.

For a long time, studies to prevent the formation of biofilms focused on the initial steps of biofilm formation, including the adhering and maturation of the biofilm. The concentrations of drugs needed to inhibit the formation of a biofilm are generally much lower than those required to destroy or perturb a preformed biofilm.

The main advantage of ANP evidenced herein by the inventors, is that use thereof at 0.1 μM for 2 h, even 30 minutes, is sufficient to disperse about 80% of the biofilm structure. This is of particular interest especially with a view to use in synergy with antibiotics.

This effect of ANP on a P. aeruginosa biofilm is greater than the inhibitory effect on formation observed previously with CNP, and exhibits an additional advantage since CNP slightly increased bacterial virulence and cytotoxicity by activating bacterial quorum-sensing systems.

The inventors have shown here that ANP, despite its strong anti-biofilm action, does not kill bacteria and does not increase the cytotoxicity of P. aeruginosa in cultured human pulmonary cells. The fact that the bacteria are not killed is of particular interest since this prevents the emergence of resistant strains.

### Example 2: Dose-Dependent Effect of ANP on Preformed Biofilms of P. aeruginosa

This example shows that the action of ANP alone on preformed biofilms of P. aeruginosa is dependent upon the dose used and can be seen on and after 0.3 ng/ml.

### Material and Methods

The material and methods used in this example are the same as those described above in Example 1 (Destruction of the P. aeruginosa biofilm under dynamic conditions).

### Results

The inventors studied the potential anti-biofilm activity of ANP on an established biofilm of P. aeruginosa at different ANP concentrations: 0.3 ng/ml, 3 ng/ml and 30 ng/ml, comparing with the activity given in Example 1 ([ANP]=300 ng/ml or 0.1 μM), after exposure for 2 h or 30 min.

The inventors observed that the biofilm is strongly dispersed by ANP on and after the lowest concentration of 0.3 ng/ml and as soon as after exposure for 30 min.

The results obtained are given in the following Table.

| | Biovolume | | Biovolume | |
|---|---|---|---|---|
| Experimental | (2 h) | % Inhibition | (30 min) | % Inhibition |
| conditions | (μm³/μm²) | (2 h) | (μm³/μm²) | (30 min) |
| PA14 non-treated | 21.1 ¶ | | 22.19 | |
| ANP (0.3 ng/ml) | 10.6 | 45.1 (**) | 9.5 | 57.5 (*) |
| ANP (3 ng/ml) | 6.3 | 65.9 (***) | 8.8 | 59.1 (*) |
| ANP (30 ng/ml) | 4.4 | 72.5 (***) | 6.7 | 69.0 (*) |
| ANP (300 ng/ml) | 4.9 | 81.4 (***) | 4.4 | 80.2 (*) |

¶: Mean of all controls;
(*): p < 0.05 vs non-treated biofilms;
(**): p < 0.01 vs non-treated biofilms;
(***): p < 0.001 vs non-treated biofilms.

This example therefore confirms the advantage of ANP which is active on and after a very low concentration and starting from an exposure time of 30 min.

### Example 3: Effect of Osteocrin on Preformed Biofilms of P. aeruginosa

This example shows the action of osteocrin alone on preformed biofilms of P. aeruginosa at a dose of 10⁻⁸ M.

### Material and Methods

The material and methods used in this example are the same as those described above in Example 1 (Destruction of the P. aeruginosa biofilm under dynamic conditions).

### Results

The inventors studied the potential anti-biofilm activity of osteocrin on an established P. aeruginosa biofilm at a concentration of 10⁻⁸ M and after exposure for 2 h.

The inventors observed that the biofilm was strongly and significantly dispersed by osteocrin.

| | Biovolume | |
|---|---|---|
| | (2 h) | % Inhibition |
| Experimental conditions | (μm³/μm²) | (2 h) |
| PA14 non-treated (control) | 29.8 | |
| Osteocrin (10⁻⁸ M) | 12.1 | 59.4 (***) |

### Example 4: Effect of the Lebetin 2α Peptide on Preformed Biofilms of P. aeruginosa

This example shows the action of the lebetin L2 alpha peptide alone on preformed biofilms of P. aeruginosa, at a dose of 10⁻⁸ M.

### Material and Methods

The material and methods used in this example are the same as those described above in Example 1 (Destruction of the P. aeruginosa biofilm under dynamic conditions).

### Results

The inventors studied the potential anti-biofilm activity of the lebetin L2 alpha peptide on an established biofilm of P. aeruginosa at a concentration of 10⁻⁸ M and after exposure for 2 h.

The inventors observed that the biofilm was strongly and significantly dispersed by the lebetin L2 alpha peptide.

| | Biovolume | |
|---|---|---|
| | (2 h) | % Inhibition |
| Experimental conditions | (μm³/μm²) | (2 h) |
| PA14 non-treated (control) | 29.7 | |
| Lebetin L2 alpha (10⁻⁸ M) | 8.9 | 70.2 (***) |

### Example 5: Effect of the Lebetin 1β Peptide on Preformed Biofilms of P. aeruginosa

This example shows the action of the lebetin L1 beta peptide alone on preformed biofilms of P. aeruginosa at a dose of 10⁻⁸ M.

### Material and Methods

The material and methods used in this example are the same as those described above in Example 1 (Destruction of the P. aeruginosa biofilm under dynamic conditions).

### Results

The inventors studied the potential anti-biofilm activity of the lebetin L1 beta peptide on an established biofilm of P. aeruginosa at a concentration of 10⁻⁸ M and after 2 h exposure.

The inventors observed that the biofilm was strongly dispersed by the lebetin L1 beta peptide.

| | Biovolume | |
|---|---|---|
| | (2 h) | % Inhibition |
| Experimental conditions | (μm³/μm²) | (2 h) |
| PA14 non-treated (control) | 21.0 | |
| Lebetin L1 beta (10⁻⁸ M) | 6.9 | 67.0 |

### Example 6: Effect of a Combination of ANP and Tobramycin on Preformed Biofilms of P. aeruginosa

This example shows the synergic action of the ANP+tobramycin combination on preformed biofilms of P. aeruginosa.

### Material and Methods

The material and methods used in this example are the same as those described above in Example 1, with the following specifications:

To study the impact of ANP in combination with tobramycin on 24 h preformed biofilms (as described in Example 1), 300 μl of a mixture of ANP (1 nM) and tobramycin (50 μg/ml) or 300 μl of tobramycin alone (control conditions) were injected into different channels of the flow chamber. Treatment of the preformed biofilm was conducted for 2 h at 37° C. under static conditions. After the 2 h, the flow of medium was again applied for 15 min to remove any cells which may have detached from the biofilm under action of the treatment.

The biofilms were observed under a confocal laser scanning microscope as described in Example 1. To test the membrane integrity of the bacteria, a mixture of 5 μM SYTO 9 Green and 0.3 μM propidium iodide (PI) was used (Live/Dead BacLight Bacterial Viability Kit, Invitrogen).

### Results

The inventors studied the potential anti-biofilm activity of the combination ANP (10⁻⁹ M; 3 ng/ml)+tobramycin (50 μg/ml or 10 μg/ml) on an established biofilm of P. aeruginosa.

The biovolume of the biofilm was determined and the results are given in the Tables below.

| | Biovolume | |
|---|---|---|
| Experimental conditions | (μm³/μm²) | % Inhibition |
| PA14 non-treated | 13.5 | |
| Tobramycin (50 μg/ml) | 3.26 | 75.9 |
| ANP (10⁻⁹ M) | 4.53 | 66.4 |
| Tobramycin (50 μg/ml) + ANP (10⁻⁹ M) | 0.5 | 96.3 |

| | Biovolume | |
|---|---|---|
| Experimental conditions | (μm³/μm²) | % Inhibition |
| PA14 non-treated | 13.5 | |
| Tobramycin (10 μg/ml) | 2.9 | 78.5 |
| ANP (10⁻⁹ M) | 4.53 | 66.4 |
| Tobramycin (10 μg/ml) + ANP (10⁻⁹ M) | 0.7 | 94.8 |

Under these conditions, the inventors observed that the biofilm was strongly dispersed by the combination ANP (1 nM)+tobramycin (50 μg/ml), which acts in synergy to obtain 96.3% destruction of the biofilm. Similarly, the combination ANP (1 nM)+tobramycin (10 μg/ml), acts in synergy to obtain 94.8% destruction of the biofilm.

### Example 7: Effect of an ANP and Ciprofloxacin Combination on Preformed Biofilms of P. aeruginosa

This example shows the synergic action of the ANP+ciprofloxacin combination on preformed biofilms of P. aeruginosa.

### Material and Methods

To study the impact of ANP in combination with ciprofloxacin on 24 h preformed biofilms (as described in Example 1), 300 μl of a mixture of ANP (10 nM) and ciprofloxacin (0.01 μg/ml or 0.04 μg/ml) or 300 μl of ciprofloxacin alone (control conditions) were injected into different channels of the flow chamber. Treatment of the preformed biofilm was conducted for 2 h at 37° C. under static conditions. After the 2 h, the flow of medium was again applied for 15 min to remove any cells which may have detached from the biofilm under action of the treatment.

As described in Example 1, the biofilms were observed under a confocal laser scanning microscope after being stained for 15 min with 5 μM of Syto9 Green fluorochrome (Invitrogen).

### Results

The inventors studied the potential anti-biofilm activity of the combination ANP (10⁻⁸ M; 30 ng/ml)+ciprofloxacin (0.04 μg/ml or 0.01 μg/ml) on an established biofilm of P. aeruginosa.

The biovolume of the biofilm was determined and the results are given in the Tables below.

| | Biovolume | |
|---|---|---|
| Experimental conditions | (μm³/μm²) | % Inhibition |
| PA14 non-treated | 12.6 | |
| Ciprofloxacin (0.04 μg/ml) | 0.7 | 99.3 |
| Ciprofloxacin (0.04 μg/ml) + ANP (10⁻⁸ M) | 0.5 | 99.5 |

| | Biovolume | |
|---|---|---|
| Experimental conditions | (μm³/μm²) | % Inhibition |
| PA14 non-treated | 15.5 | |
| Ciprofloxacin (0.01 μg/ml) | 1.55 | 90.0 |
| Ciprofloxacin (0.01 μg/ml) + ANP (10⁻⁸ M) | 0.47 | 97.0 |

Under these conditions, the inventors observed that the biofilm was even more strongly dispersed by the combination ANP+ciprofloxacin, compared to treatment with the ciprofloxacin antibiotic alone.

Therefore, the combination with ANP allows 97% destruction of the biofilm to be obtained in combination with a ciprofloxacin concentration of 0.01 μg/ml.

### Example 8: Effect of a Combination of ANP and Colistin on Preformed Biofilms of P. aeruginosa

This example shows the synergic action of the ANP+colistin combination on preformed biofilms of P. aeruginosa.

### Material and Methods

To study the impact of ANP in combination with colistin on 24 h preformed biofilms (as described in Example 1), 300 μl of a mixture of ANP (10 nM) and colistin (1 μg/ml) or 300 μl of colistin alone (control conditions) were injected into different channels of the flow chamber. Treatment of the preformed biofilm was conducted for 2 h at 37° C. under static conditions. After the 2 h, the flow of medium was again applied for 15 min to remove any cells which may have detached from the biofilm under action of the treatment.

As described in Example 1, the biofilms were observed under a confocal laser scanning microscope, after being stained for 15 min with 5 μM of Syto9 Green fluorochrome (Invitrogen).

### Results

The inventors studied the potential anti-biofilm activity of the combination ANP (10⁻⁸ M; 30 ng/ml)+colistin (1 μg/ml) on an established biofilm of P. aeruginosa.

The biovolume of the biofilm was determined and the results are given in the Tables below.

| | Biovolume | |
|---|---|---|
| Experimental conditions | (μm³/μm²) | % Inhibition |
| PA14 non-treated | 18.5 | |
| ANP (10⁻⁸ M) | 4.9 | 73.5 |
| Colistin (1 μg/ml) | 2.1 | 88.6 |
| Colistin (1 μg/ml) + ANP (10⁻⁸ M) | 0.5 | 97.3 |

Under these conditions, the inventors observed that the biofilm was even more strongly dispersed by the combination ANP+colistin (1 μg/ml), compared to treatment with the colistin antibiotic alone or treatment with ANP alone.

Therefore, the combination with ANP allows 97.3% destruction of the biofilm to be obtained, in combination with a colistin concentration of 1 μg/ml.

### Example 9: Effect of a Combination of ANP and Imipenem on Preformed Biofilms of P. aeruginosa

This example shows the synergic action of the ANP+Imipenem combination on preformed biofilms of P. aeruginosa.

### Material and Methods

To study the impact of ANP in combination with Imipenem on 24 h preformed biofilms (as described in Example 1), 300 μl of a mixture of ANP (10 nM) and Imipenem (0.5 μg/ml) or 300 μl of Imipenem alone (control conditions) were injected into different channels of the flow chamber. Treatment of the preformed biofilm was conducted for 2 h at 37° C. under static conditions. After the 2 h, the flow of medium was again applied for 15 min to remove any cells which may have detached from the biofilm under action of the treatment.

As described in Example 1, the biofilms were observed under a confocal laser scanning microscope after being stained for 15 min with 5 μM of Syto9 Green fluorochrome (Invitrogen).

### Results

The inventors studied the potential anti-biofilm activity of the combination ANP (10⁻⁸ M; 30 ng/ml)+Imipenem (0.5 μg/ml) on an established biofilm of P. aeruginosa.

The biovolume of the biofilm was determined and the results are given in the Tables below.

| | Biovolume | |
|---|---|---|
| Experimental conditions | (μm³/μm²) | % Inhibition |
| PA14 non-treated | 28.0 | |
| ANP (10⁻⁸ M) | 6.9 | 75.4 |
| Imipenem (0.5 μg/ml) | 7.0 | 75.0 |
| Imipenem (0.5 μg/ml) + ANP (10⁻⁸ M) | 0.6 | 97.9 |

Under these conditions, the inventors observed that the biofilm was even more strongly dispersed by the ANP+Imipenem (0.5 μg/ml) combination, compared to treatment with the Imipenem antibiotic alone or with ANP treatment alone.

Therefore, the combination with ANP allows 97.9% destruction of the biofilm to be obtained in combination with an Imipenem concentration of 0.5 μg/ml.

### Example 10: Effect of a Combination of ANP and Polymyxin B on Preformed Biofilms of P. aeruginosa

This example shows the synergic action of the ANP+Polymyxin B combination on preformed biofilms of P. aeruginosa.

### Material and Methods

To study the impact of ANP in combination with polymyxin B on 24 h preformed biofilms (as described in Example 1), 300 μl of a mixture of ANP (10 nM) and Polymyxin B (4 μg/ml) or 300 μl of Polymyxin B alone (control conditions) were injected into different channels of the flow chamber. Treatment of the preformed biofilm was conducted for 2 h at 37° C. under static conditions. After the 2 h, the flow of medium was again applied for 15 min to remove any cells which may have detached from the biofilm under action of the treatment.

As described in Example 1, the biofilms were observed under a confocal laser scanning microscope after being stained for 15 min with 5 μM of Syto9 Green fluorochrome (Invitrogen).

### Results

The inventors studied the potential anti-biofilm activity of the combination ANP (10⁻⁸ M; 30 ng/ml)+Polymyxin B (4 μg/ml) on an established biofilm of P. aeruginosa.

The biovolume of the biofilm was determined and the results are given in the Tables below.

| | Biovolume | |
|---|---|---|
| Experimental conditions | (μm³/μm²) | % Inhibition |
| PA14 non-treated | 18.2 | |
| ANP (10⁻⁸ M) | 4.5 | 75.3 |
| Polymyxin B (4 μg/ml) | 7.4 | 59.3 |
| Polymyxin B (4 μg/ml) + ANP (10⁻⁸ M) | 3.0 | 83.5 |

Under these conditions, the inventors observed that the biofilm was even more strongly dispersed by the ANP+Polymyxin B (4 μg/ml) combination, compared to treatment with the Polymyxin B antibiotic alone or with ANP treatment alone.

Therefore, the combination with ANP allows 83.5% destruction of the biofilm to be obtained in combination with a Polymyxin B concentration of 4 μg/ml.

### Example 11: Effect of Sequential Treatment with ANP Followed by Tobramycin on Preformed Biofilms of P. aeruginosa

This example shows the sequential action of treatment with ANP followed by treatment with tobramycin on preformed biofilms of P. aeruginosa.

### Material and Methods

The material and methods used in this example are the same as those described above in Example 1, with the following specifications:

To study the impact of sequential exposure to ANP and then to tobramycin on 24 h preformed biofilms (as described in Example 1), 300 μl of ANP (1 nM) (control conditions) were injected into two channels of the flow chamber. Treatment of the biofilm was conducted for 2 h at 37° C. under static conditions. After the 2 h, the flow of medium was again applied for 15 min to remove any cells which may have detached from the biofilm under action of the treatment. Next, 300 μl of tobramycin (50 μg/ml) or 300 μl of milliQ sterile water (control conditions) were injected into the two channels of the flow chamber pre-exposed to ANP. The second treatment of the preformed biofilm was again conducted for 2 h at 37° C. under static conditions. After the 2 h, the flow of medium was again applied for 15 min to remove any cells which may have detached from the biofilm under action of the treatment.

As described in Example 1, the biofilms were observed under a confocal laser scanning microscope after being stained for 15 min with 5 μM of Syto9 Green fluorochrome (Invitrogen).

### Results

The inventors studied the potential anti-biofilm activity of sequential exposure to ANP (10⁻⁹ M; 3 ng/ml) followed by tobramycin (50 μg/ml) on an established biofilm of P. aeruginosa.

The biovolume of the biofilm was determined and the results are given in the Tables below.

| | Biovolume | |
|---|---|---|
| Experimental conditions | (μm³/μm²) | % Inhibition |
| PA14 non-treated | 13.1 | |
| ANP (10⁻⁹ M) | 4.1 | 68.7 |
| ANP (10⁻⁹ M) (2 h) then Tobramycin (50 μg/ml) (2 h) | 0.7 | 94.7 |

Under these conditions, the inventors observed that the biofilm was even more strongly dispersed by twofold sequential treatment with ANP (10⁻⁹ M) (2 h) then tobramycin (50 μg/ml) (2 h) compared to treatment with ANP alone.

Therefore, sequential treatment with ANP (2 h) then tobramycin (2 h) allows 94.7 destruction of the biofilm to be obtained.

1-10. (canceled)

11. A method of therapeutically treating a bacterial infection associated with a bacterial biofilm in a subject in need thereof, the method comprising:
administering a therapeutically effective amount of a natriuretic peptide to the subject to disperse the bacterial biofilm, wherein the natriuretic peptide is selected from the group consisting of (i) osteocrin, an osteocrin propeptide or osteocrin derivative, (ii) a lebetin, a lebetin fragment, or a lebetin or lebetin fragment derivative, and (iii) an ANP peptide, ANP propeptide or ANP peptide derivative;
thereby therapeutically treating the bacterial infection associated with the bacterial biofilm in the subject.

12. The method according to claim 11, wherein the bacterial infection is an infection with Pseudomonas aeruginosa.

13. The method according to claim 11, wherein the subject suffers from a chronic respiratory pathology.

14. The method according to claim 11, wherein the subject suffers from cystic fibrosis.

15. The method according to claim 11, wherein the subject has an implant or prosthesis, or suffers from major burns or a surface infection.

16. The method according to claim 11 further comprising administering a therapeutically effective amount of an antibiotic in combination with the natriuretic peptide.

17. The method according to claim 16, wherein the antibiotic is an aminoglycoside antibiotic or a quinolone.

18. The method according to claim 11, further comprising administering a therapeutically effective amount of at least one other additional natriuretic peptide in combination with the first natriuretic peptide.

19. A pharmaceutical composition comprising:
(a) a natriuretic peptide selected from the group consisting of (i) osteocrin, an osteocrin propeptide or osteocrin derivative, (ii) a lebetin, a lebetin fragment, or a lebetin or lebetin fragment derivative, and (iii) an ANP peptide, ANP propeptide or ANP peptide derivative; and
(b) an antibiotic.

20. A pharmaceutical antibacterial combination for therapeutically treating a bacterial infection associated with a bacterial biofilm in a subject in need thereof, the pharmaceutical antibacterial combination comprising:
(a) a natriuretic peptide selected from the group consisting of (i) osteocrin, an osteocrin propeptide or osteocrin derivative, (ii) a lebetin, a lebetin fragment, or a lebetin or lebetin fragment derivative, and (iii) an ANP peptide, ANP propeptide or ANP peptide derivative; and
(b) an antibiotic for simultaneous, separate, or sequential use with the natriuretic peptide.

21. An in vitro or ex vivo method to disperse a bacterial biofilm, the method comprising:
applying to the bacterial biofilm a natriuretic peptide selected from the group consisting of (i) osteocrin, an osteocrin propeptide or osteocrin derivative, (ii) a lebetin, a lebetin fragment, or a lebetin or lebetin fragment derivative, and (iii) an ANP peptide, ANP propeptide or ANP peptide derivative;
thereby dispersing the bacterial biofilm.


SEQUENCE LISTING (9 sequences)

SEQ ID NO: 1 - protein, 28 aa, Homo sapiens
SLRRSSCFGGRMDRIGAQSGLGCNSFRY

SEQ ID NO: 2 - protein, 32 aa, Homo sapiens
TAPRSLRRSSCFGGRMDRIGAQSGLGCNSFRY

SEQ ID NO: 3 - protein, 126 aa, Homo sapiens
NPMYNAVSNADLMDFKNLLDHLEEKMPLEDEVVPPQVLSEPNEEAGAALSPLPEVPPWTGEVSPAQRDGGALGRGPWDSSDRSALLKSKLRALLTAPRSLRRSSCFGGRMDRIGAQSGLGCNSFRY

SEQ ID NO: 4 - protein, 151 aa, Homo sapiens
MSSFSTTTVSFLLLLAFQLLGQTRANPMYNAVSNADLMDFKNLLDHLEEKMPLEDEVVPPQVLSEPNEEAGAALSPLPEVPPWTGEVSPAQRDGGALGRGPWDSSDRSALLKSKLRALLTAPRSLRRSSCFGGRMDRIGAQSGLGCNSFRY

SEQ ID NO: 5 - protein, 50 aa, Homo sapiens
SFSGFGSPLDRLSAGSVDHKGKQRKVVDHPKRRFGIPMDRIGRNRLSNSR

SEQ ID NO: 6 - protein, 106 aa, Homo sapiens
VDVTTTEAFDSGVIDVQSTPTVREEKSATDLTAKLLLLDELVSLENDVIETKKKRSFSGFGSPLDRLSAGSVDHKGKQRKVVDHPKRRFGIPMDRIGRNRLSNSRG

SEQ ID NO: 7 - protein, 133 aa, Homo sapiens
MLDWRLASAHFILAVTLTLWSSGKVLSVDVTTTEAFDSGVIDVQSTPTVREEKSATDLTAKLLLLDELVSLENDVIETKKKRSFSGFGSPLDRLSAGSVDHKGKQRKVVDHPKRRFGIPMDRIGRNRLSNSRG

SEQ ID NO: 8 - protein, 38 aa, Macrovipera lebetina
GDNKPPKKGPPNGCFGHKIDRIGSHSGLGCNKVDDNKG

SEQ ID NO: 9 - protein, 12 aa, Macrovipera lebetina
DNKPPKKGPPNG